(19) United States
(12) Patent Application Publication
(10) Pub. No.: US 2025/0352451 A1
(43) Pub. Date: Nov. 20, 2025

(54) COSMETIC PRODUCT FOR TEMPORARILY SHAPING KERATIN-CONTAINING FIBERS, HAVING CARE EFFECT

(71) Applicant: HENKEL AG & CO. KGAA, Düsseldorf (DE)

(72) Inventors: Thorsten KNAPPE, Schenefeld (DE); Pamela KAFTAN, Hamburg (DE); Alexandra GROSSDORF, Schenefeld (DE); Anja THAMMASIRI, Klein Nordende (DE)

(21) Appl. No.: 18/871,441

(22) PCT Filed: Feb. 23, 2023
(86) PCT No.: PCT/EP2023/054557

§ 371 Date: Dec. 3, 2024

(30) Foreign Application Priority Data
Jun. 15, 2022 (DE) 10 2022 115 071.2

(51) Int. Cl. A61K 8/73; A61Q 5/06

(52) U.S. Cl. CPC A61K 8/73; A61Q 5/06; A61K 2800/592; A61K 2800/95

(57) ABSTRACT

A cosmetic product for temporarily shaping keratin-containing fibers is described. The cosmetic product includes at least one non-fully synthetic film-forming agent and at least one care agent. Use the cosmetic product for caring for and temporarily shaping keratin-containing fibers, and a method for temporarily shaping keratin-containing fibers using the cosmetic product are also described.

The present invention relates to a cosmetic product for temporarily shaping keratin-containing fibers, comprising at least one non-fully synthetic film-forming agent and at least one care agent, and to the use of the product for caring for and temporarily shaping keratin-containing fibers, and to a method for temporarily shaping keratin-containing fibers using the cosmetic product according to the invention.

Cosmetic products which permanently or temporarily shape hair play an important role in cosmetics. Temporary shaping, which is intended to provide a good hold without impairing the healthy appearance of the hair, such as its shine, can be achieved, for example, by hair sprays, hair waxes, hair gels, hair foams, hairdryers, etc.

The most important property of a product for temporarily shaping keratin-containing fibers is that of providing the treated fibers with the strongest and longest possible hold in the shape created. If the keratin-containing fibers are human hair, this is also referred to as strong hairstyle hold, or a high degree of hold of the cosmetic product. Hairstyle hold is substantially determined by the type and quantity of polymers used.

Numerous cosmetic products which provide temporary shaping are known from the prior art. Corresponding products usually contain synthetic polymers as shaping components. Said products, however, cause the hair to dry out. Particularly in the case of wavy or curly hair, hold is not the sole focus of cosmetic products; rather, a combination of hold and care is desirable.

The problem addressed by the present invention is therefore to provide a cosmetic product which is suitable for temporarily shaping keratinous fibers and which, at the same time, has a care effect, i.e., does not make the keratinous fibers appear dried out or brittle. Correspondingly, styling properties should be aligned with consumer needs, such that the hold is sufficient, but the keratinous fibers do not appear brittle or “crunchy”.

At the same time, there is an increasing demand for sustainable products, in which the substances used are natural substances, or at least modified natural substances. This is intended to reduce the carbon footprint of the product.

At the same time, the product is intended to be usable as a foam, if possible, since a foam allows for particularly good application, since the foam can be distributed uniformly over the keratinous fiber and the application is particularly user-friendly.

Surprisingly, it has been found that a combination of certain care agents with non-fully synthetically produced film-forming agents (non-fully synthetic film-forming agents) in a cosmetic product in the form of a foam solves this problem. In a first embodiment, the problem addressed by the present invention is achieved by a cosmetic product for temporarily shaping and caring for keratin-containing fibers, in particular human hair, which is applied as a foam, comprising at least one non-fully synthetically produced film-forming agent, a cosmetic carrier, and at least one care agent, the care agent being selected from the group consisting of shea butter, mango butter, cocoa butter, avocado butter, shea oil, capuacu butter, illipe nut butter, kokum butter, sal butter, murumuru butter, olive butter, aloe butter, almond butter, tucuma butter, ghee (Asian clarified butter), coconut oil and organic nilotica shea (a certain variety of shea butter), and mixtures of two, three or more of the above.

According to the invention, the cosmetic product can therefore contain one, two, three or more care agents. The cosmetic product according to the invention preferably contains one, two or three care agents, in particular one or two. In particular, the care agent is selected from shea butter, mango butter, cocoa butter, avocado butter, shea oil, and mixtures of two or more of the above. Surprisingly, it is possible to obtain a cosmetic product which provides good hold despite having a high care agent content.

The cosmetic product according to the invention preferably contains the care agent in a proportion of from 0.1 wt. % to 10 wt. %, in particular from 0.2 wt. % to 8 wt. %, preferably from 0.5 wt. % to 6 wt. %, particularly preferably from 0.7 wt. % to 5 wt. %, more particularly preferably from 1 wt. % to 4 wt. %, particularly from 1.5 wt. % to 3 wt. %, in each case based on the total weight of the cosmetic product.

Surprisingly, it has been found that it is possible to provide a cosmetic product for temporary shaping in which the care and hold properties are combined in a way that corresponds to the needs of the consumer. A cosmetic product can thus be provided which not only offers hold but at the same time cares for the hair.

Keratin-containing fibers are understood in the context of the present invention to mean fur, wool, feathers, and hair, in particular human hair. This is particularly preferably understood to be textured human hair, i.e. curly (coily, kinky) or wavy hair. Particularly in the case of strongly textured hair, also known as coily or kinky, the desire for a high proportion of care ingredients is great, since the hair quickly becomes brittle.

According to the invention, the cosmetic product furthermore comprises at least one non-fully synthetically produced film-forming agent. Film-forming agents are understood to be polymers which leave behind a continuous film on the skin, hair or nails upon drying. Film-forming agents of this kind can be used in a wide variety of cosmetic products, such as face masks, make-up, hair setting products, hair sprays, hair gels, hair waxes, hair masques, shampoos or nail polishes. Preference is given to polymers which possess a sufficient solubility in water, alcohol or water/alcohol mixtures. Corresponding solutions can thus be produced which can be used or further processed in a simple manner.

Within the scope of the present invention, a film-forming agent is understood to mean, in particular, polymers which, when used in 0.05 to 20 wt. % (based on the total weight of the cosmetic product) of aqueous, alcoholic, or aqueous-alcoholic solution, are able to be deposited on the hair in the form of a transparent polymer film which makes it possible to temporarily bond hair fibers to one another.

Film-forming agents are known from the prior art. These are also distinguished with regard to their origin. Usually, fully synthetically produced film-forming agents are used in cosmetic products. Within the scope of the present invention, a film-forming agent is to be considered fully synthetically produced if all of the monomers which make up the film-forming agent are conventionally produced synthetically. The film-forming agent is to be regarded as fully synthetically produced even if a monomer was obtained from biogas or other renewable sources, for example.

In contrast to this, the non-fully synthetically produced film-forming agents are those which are based on a naturally occurring substance. According to the invention, said naturally occurring substance (natural substance) can be chemically modified. These film-forming agents based on natural substances are referred to, in the present case, as non-fully synthetically produced film-forming agents.

The cosmetic product according to the invention contains at least one non-fully synthetically produced film-forming agent. It can furthermore contain at least one fully synthetically produced film-forming agent. According to the invention, the cosmetic product therefore comprises one, two, three or more different non-fully synthetically produced film-forming agents. Additionally, it may have one, two, three or more non-fully synthetically produced film-forming agents.

In a further preferred embodiment, the cosmetic product according to the invention is substantially free of fully synthetically produced film-forming agents. Surprisingly, it has been found that a cosmetic product which is substantially free of fully synthetic film-forming agents is not necessarily associated with losses in styling properties. In the context of the present invention, based on the total weight of the product, the cosmetic product can comprise up to 5 wt. %, in particular up to 3 wt. %, preferably up to 2 wt. %, preferably 1 wt. % or less of fully synthetic polymers, which are film-forming agents in the conventional sense, in order to be “substantially” free from fully synthetic film-forming agents. Preferably, the cosmetic product can also be free of fully synthetic film-forming agents, i.e. the cosmetic product comprises 0 wt. % of fully synthetic film-forming agents.

Non-fully synthetically produced film-forming agents, within the meaning of the present invention, are polymers or copolymers which are based on a natural substance. According to the invention, these natural substances can still be chemically modified.

Suitable natural substances are, for example, saccharides (mono-, di- and polysaccharides) or biopolymers (polymers synthesized in the cell of a living being).

Suitable saccharides are, for example, glucose, fructose, sucrose, lactose, maltose, cellulose or starch. Plant rubbers (polysaccharides of vegetable origin), such as guar gum (guar), agar, carrageenan, Acacia senegal gum or others are also suitable in the present case. A suitable biopolymer is chitosan. These compounds, in particular chemically modified, can be used as film-forming agents, alone or in mixtures, in the cosmetic product according to the invention.

Suitable cellulose derivatives are in particular hydroxypropyl cellulose, having a molecular weight of 30,000 to 50,000 g/mol, cationic cellulose derivatives, in particular those which result from a reaction of hydroxyethyl cellulose with a dimethyl diallylammonium reactant (in particular dimethyl diallylammonium chloride).

Also suitable are polysaccharides, which are in particular obtained from starch, for example the polysaccharide having the INCI name pullulan. Also suitable are, for example, potato starch, or a starch modified by non-ionic propylene oxide, as described in DE 10 2017 222 854 A1 or DE 10 2018 218 270 A1.

Starch is a reserve carbohydrate which is stored by many plants in the form of starch grains (granules), usually 1 to 200 μm in size, in different plant parts, for example in tubers or roots, cereal seeds, fruits, and in the pith. A useable, non-ionic, propylene oxide-modified starch can be derived from starch from potatoes, corn, rice, peas, acorns, chestnuts, barley, wheat, bananas, sago, millet, sorghum, oats, barley, rye, beans, sweet potato, arrowroot or manioc. Particularly pronounced advantageous effects are achieved by non-ionic, propylene oxide-modified tapioca starch or non-ionic, propylene oxide-modified potato starch, or by mixtures of both aforementioned starches. The cosmetic product very particularly preferably contains at least one non-ionic, propylene oxide-modified potato starch. A non-ionic propylene oxide-modified starch is thus understood to mean a reaction product of a starch and propylene oxide. Pea starch or a modified pea starch can also preferably be used, the modified pea starch preferably being a non-ionic, propylene oxide-modified pea starch.

Furthermore, guar hydroxypropyltrimonium chloride can be used according to the invention as a non-fully synthetic film-forming agent, if it has film-forming properties.

Chitosans are biopolymers and belong to the group of hydrocolloids. Considered chemically, these are partially deacetylated chitins of differing molecular weight. For the preparation of the chitosans, chitin is used, preferably the shell residues of crustaceans, which are available in large amounts as cheap raw materials. The chitin is usually first deprotonated by the addition of bases, demineralized by the addition of mineral acids, and finally deacetylated by the addition of strong bases. Alternatively, the deacetylation can also take place enzymatically. The molecular weights can be distributed over a wide spectrum. Types which have an average molecular weight (weight average) of from 800,000 to 1,200,000 daltons, a Brookfield viscosity (1 wt % in glycolic acid) below 5000 mPas, a degree of deacetylation in the range from 80 to 88%, and an ash content of less than 0.3 wt % are preferably used.

In addition to the chitosans, cationically derivatized chitosans (such as quaternizing products) or alkoxylated chitosans can also be used.

Suitable chitosans are freely available commercially, for example under the trade names Hydagen® CMF (1 wt. % of active substance in an aqueous solution with 0.4 wt. % of glycolic acid, molecular weight 500,000 to 5,000,000 g/mol; Cognis), Hydamer® HCMF (80% deacetylated chitosan, molecular weight 50,000 to 1000000 g/mol, Chitinor, formerly Cognis), Kytamer® PC (about 80 wt. % of active substance chitosan pyrrolidone carboxylate (INCI name: Chitosan PCA), Amerchol), Chitolam® NB/101 and Chitosan 90/100/A1 (about 90% deacetylated chitosan, BioLog Heppe).

Very particularly preferably, the cosmetic products can contain neutralization products of chitosan, having at least one acid as chitosan derivative(s). In the context of the present invention, the neutralization product is any product which is produced by conversion of chitosan using an organic carboxylic acid, i.e. the neutralization product can be prepared separately before the preparation of the cosmetic product, or the neutralization product can be formed in situ during the production of the cosmetic product.

According to a preferred embodiment of the present invention, the organic carboxylic acid is to be selected from formic acid, acetic acid, citric acid, lactic acid, pyrrolidone carboxylic acid, tartaric acid, glycolic acid, nicotinic acid, hydroxyisobutyric acid, hydroxyisovaleric acid, or mixtures of these acids, in particular lactic acid. This neutralization product can be prepared, for example, in an aqueous medium, by adding chitosan and the corresponding organic carboxylic acid, or a portion of the corresponding organic carboxylic acid.

According to a preferred embodiment of the present invention, the weight ratio of chitosan to the organic acid is between 2:1 and 1:10, preferably between 1:1 and 1:6, even more preferably between 1:2 and 1:4. In this more preferred embodiment, the total weight of the chitosan used is based on the total weight of the organic acid in the cosmetic product. A particularly preferred organic acid is lactic acid. Lactic acid is a solubility enhancer. Used in the above weight ratios, it achieves particularly advantageous styling effects and gives the hair an exceptional hairstyle hold. Excellent styling properties can be obtained, in particular during so-called curl retention tests, which are carried out at high atmospheric humidity.

Examples of film-forming agents which are usually produced fully synthetically comprise in particular copolymers based on acrylate or methacrylate, in particular those which comprise acrylic acid, methacrylic acid, alkyl (meth) acrylate, hydroxyalkyl (meth) acrylate and/or maleic anhydride as a monomer component, and copolymers which comprise N-vinylpyrrolidone, alkyl vinyl ethers having alkyl groups comprising 1 to 18 carbon atoms and/or vinyl esters of carboxylic acids having 2 to 18 carbon atoms as monomer components, in particular polyvinylpyrrolidone/vinyl acetate copolymers; and also polyvinylpyrrolidone; wherein copolymers are understood to be polymers consisting of two or more different monomers, and all alkyl groups of the monomers mentioned can also be branched alkyl groups and/or alkyl groups substituted with functional groups. Corresponding film-forming agents are usually regarded as fully synthetic film-forming agents. Corresponding fully synthetic film-forming agents are well known to a person skilled in the art.

The product according to the invention contains film-forming polymers (film-forming agents), preferably in a proportion of from 0.05 wt. % to 30 wt. %, in particular from 0.10 wt. % to 20 wt. %, preferably from 0.15 wt. % to 15 wt. %, based on the total weight of the cosmetic product. In a preferred embodiment, the cosmetic product is substantially free of fully synthetic film-forming polymers, so that in this embodiment, the cosmetic product contains non-fully synthetic film-forming polymers in a proportion of from 0.05 wt. % to 30 wt. %, in particular 0.15 wt. % to 20 wt. %, preferably 0.2 wt. % to 15 wt. %, based on the total weight of the cosmetic product. The specified amounts are in each case based on all polymers present when mixtures of a plurality of film-forming polymers (film-forming agents) are contained.

According to a preferred embodiment of the present invention, the cosmetic product contains a care polymer in small amounts, which polymer is particularly preferably a cationic homopolymer. Cationic polymers are understood to be polymers which comprise a group, in the main chain and/or side chain, that can be “temporarily” or “permanently” cationic. According to the invention, the term “permanently” cationic refers to polymers that comprise a cationic group irrespective of the pH of the product. These are generally polymers that contain a quaternary nitrogen atom, for example in the form of an ammonium group. Preferred cationic groups are quaternary ammonium groups.

In particular homopolymers in which the quaternary ammonium group is attached to a homopolymer main chain constructed from acrylamide via a C₁₋₄ alkylene group, the three alkyl groups other than the C₁₋₄ alkylene group of the ammonium group being chosen independently of one another from C₁₋₁₀ alkyl groups, have proven to be particularly suitable.

All possible physiologically acceptable anions, such as chloride, bromide, acetate, sulphate and/or phosphate, may be used to compensate for the positive polymer charge.

The product can also contain as a care component, for example, at least one protein hydrolyzate and/or a derivative thereof. Protein hydrolyzates are product mixtures that are obtained by acid-, base-, or enzyme-catalyzed degradation of proteins. The term protein hydrolyzates is also understood to mean total hydrolyzates, as well as individual amino acids and derivatives thereof, and mixtures of different amino acids. The molar weight of the usable protein hydrolyzates is between 75, the molar weight of glycine, and 200,000; the molar weight preferably being 75 to 50,000 daltons, and very particularly preferably 75 to 20,000 daltons.

Likewise suitable care agents, according to the invention, are in particular quaternary ammonium derivatives, in particular BTAC or CTAC.

The product according to the invention can furthermore contain at least one vitamin, a provitamin, a vitamin precursor, and/or one of the derivatives thereof as a care component. In this case, the vitamins, provitamins, and vitamin precursors that are usually assigned to the groups A, B, C, E, F, and H are preferred. A particularly preferred care component is D-panthenol.

The advantage of said care components is that they originate largely from natural sources, and are thus raw materials that are energy-saving and resource-saving. According to the preferred embodiment, the cosmetic product, based on the total weight, can contain 0.0005 to 3 wt. %, preferably 0.001 to 2 wt. %, and particularly preferably 0.05 to 1 wt. % of the sum of care oils and care components.

To adjust the pH, the cosmetic product can furthermore contain neutralizers or pH adjusters. According to a further preferred embodiment, the cosmetic product can additionally contain an emulsifier. Emulsifiers lead to the formation, at the phase interface, of water-resistant or oil-resistant adsorption layers, which prevent dispersed droplets from coalescing and thus stabilize the emulsion. Like surfactants, emulsifiers are therefore formed from a hydrophobic and a hydrophilic molecule part. Hydrophilic emulsifiers preferably form O/W-emulsions (oil-in-water emulsions) and hydrophilic emulsifiers preferably form W/O-emulsions (water-in-oil emulsions). Particularly preferred emulsifiers are alkoxylated fatty alcohols. The particularly preferred emulsifiers offer the advantage of being able to be obtained from natural, non-fully synthetic raw material sources.

Accordingly, in a preferred embodiment, the cosmetic product further contains at least one alkoxylated fatty alcohol.

An “alkoxylated fatty alcohol” is any fatty alcohol with a carbon chain of C₅ or more, which further comprises at least one alkoxy group. The fatty alcohol can, for example, be selected from C₉-C₁₁ fatty alcohols, C₁₂-C₁₃ fatty alcohols, C₁₂-C₁₅ fatty alcohols, C12-C16 fatty alcohols, C₁₄-C₁₅ fatty alcohols, arachidyl alcohol, behenyl alcohol, caprylic alcohol, cetearyl alcohol, cetyl alcohol, coconut alcohol, decyl alcohol, (hydrogenated) tallow alcohol, lauryl alcohol, myristyl alcohol, oleyl alcohol, palm alcohol, palm kernel alcohol, stearyl alcohol, and tridecyl alcohol.

The alkoxy group can in particular comprise ethoxy and/or propoxy and/or butoxy groups. The alkoxylated fatty alcohol is particularly preferably an ethoxylated fatty alcohol.

The at least one alkoxy group of the “alkoxylated fatty alcohol” can be derived, for example, from an alkoxylation reaction with alkylene oxide, in particular ethylene oxide and/or propylene oxide.

It is preferred that the at least one alkoxylated fatty alcohol is selected from the group consisting of ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, ceteareth-33, ceteareth-34, ceteareth-40, ceteareth-50, ceteareth-55, ceteareth-60, ceteareth-80, ceteareth-100, laureth-1, laureth-2, laureth-3, laureth-4, laureth-5, laureth-6, laureth-7, laureth-8, laureth-9, laureth-10, laureth-11, laureth-12, laureth-13, laureth-14, laureth-15, laureth-16, laureth-20, laureth-23, laureth-25, laureth-30, laureth-40, deceth-3, deceth-5, oleth-5, oleth-30, steareth-2, steareth-4, steareth-6, steareth-7, steareth-10, steareth-11, steareth-13, steareth-14, steareth-15, steareth-20, steareth-21, steareth-25, steareth-27, steareth-30, steareth-40, steareth-50, steareth-100 and mixtures thereof.

The designation ceteareth-2 is in this case, for example, a C₁₆-C₁₈ fatty alcohol having an average of 2 ethylene oxide units per molecule.

In a very particularly preferred embodiment of the cosmetic product, the at least one alkoxylated fatty alcohol comprises cetearyl alcohol and/or ceteareth 20, in particular cetearyl alcohol and ceteareth 20.

The amount of the at least one alkoxylated fatty alcohol is preferably 1 to 15 wt. % and more preferably 2 to 8 wt. %, in each case based on the total amount of the cosmetic product.

According to the invention, the cosmetic products can additionally have at least one surfactant. In general, non-ionic, anionic, cationic and ampholytic surfactants can be contained in cosmetic products. The group of the ampholytic or amphoteric surfactants comprises zwitterionic surfactants and ampholytes. It has been found to be particularly advantageous if the cosmetic products additionally contain at least one cationic surfactant for increasing hair-care properties.

It is preferably possible to use cationic surfactants of the following types: quaternary ammonium compounds, esterquats, and amidoamines. Preferred quaternary ammonium compounds are ammonium halides, in particular chlorides and bromides, such as alkyl trimethylammonium n chlorides, dialkyl dimethylammonium chlorides, and trialkyl methylammonium chlorides. The long alkyl chains of these surfactants preferably have 10 to 30 carbon atoms, such as in cetyl trimethylammonium chloride, stearyl trimethylammonium distearyl chloride, dimethylammonium chloride, lauryl dimethylammonium chloride, lauryl dimethylbenzylammonium chloride, and tricetyl methylammonium chloride. According to a preferred embodiment of the present invention, alkyl trimethylammonium salts are used, preferably C₁₂-C₂₅ alkyl trimethylammonium salts, particularly preferably C₁₆-C₂₅ alkyl trimethylammonium chlorides, in particular cetrimonium chloride (CTAC) or behentrimonium chloride (BTAC).

In a preferred embodiment of the present invention, the cosmetic product contains 0.01 to 4 wt. %, particularly preferably 0.1 to 3 wt. %, and in particular 0.2 to 2 wt. %, of the cationic surfactant, the specified amounts relating to the total weight of the cosmetic product.

The applicability of the cosmetic product can be further increased by the use of small amounts of one or more polyhydric alcohols. Preferred cosmetic products contain one or more C₃ to C₁₂ alkane-1,2-diols, more preferably C₃ to C₁₀ alkane-1,2-diols. The cosmetic product particularly preferably contains propane-1,2-diol and/or caprylyl glycol. These particularly preferred diols improve the applicability, are so-to-speak nourishing, and are suitable for forming a stable emulsion with the particularly preferred emulsifier.

In further preferred embodiments of the present invention, the cosmetic product further contains perfume and optionally contains further auxiliaries, such as means for adjusting the viscosity. These thickeners are preferably also non-synthetic, just like the film-forming agent.

The cosmetic product contains the ingredients described above in a cosmetic carrier. The cosmetic carrier is preferably an aqueous or aqueous-alcoholic carrier. The cosmetic product contains, based on its total weight, preferably at least 25 wt. %, more preferably at least 40 wt. %, and particularly preferably at least 60 wt. % of water. The cosmetic product particularly preferably comprises at least 65 wt. % of water, particularly preferably at least 70 wt. % of water or at least 75 wt. % of water.

Insofar as specified amounts are mentioned in the present application in wt. %, these refer to the active substance of the relevant substance based on the total weight of the product, unless stated otherwise.

The cosmetic products are used as a foam. For this purpose, the cosmetic products according to the invention are preferably packaged in a suitable dispensing device, which is either a compressed gas container (“aerosol container”), which is additionally filled with a propellant, or a non-aerosol container. Compressed gas containers, by means of which a product is dispensed via a valve by means of the internal gas pressure of the container, are referred to by definition as “aerosol containers”. Conversely to the aerosol definition, a container under standard pressure, by means of which a product is dispensed by a pumping or squeezing system by means of mechanical action, is defined as a “non-aerosol container”.

Particularly preferably, the cosmetic product is present as aerosol foam in an aerosol container. The cosmetic product therefore preferably additionally contains at least one propellant. In the embodiment as an aerosol foam, suitable propellants are selected, for example, from N₂O, dimethyl ether, CO₂, nitrogen, air, alkanes having 3 to 5 carbon atoms, such as propane, n-butane, isobutane, n-pentane, and isopentane, and mixtures thereof.

The amount of propellant is dependent on the type and size of the selected container in which the cosmetic product is offered and used. The amount of propellant in the cosmetic product is therefore selected such that a pressure of 5 bar to 15 bar, in particular of 7 bar to 12 bar, is produced in the container containing the cosmetic product. Such a pressure is usually selected in order to produce a good foam and to empty a container as completely as possible. Higher pressures can be used by selecting packagings having a corresponding pressure resistance, but are not common due to the higher material use required.

When conventional aerosol containers are used, aerosol foam products contain propellants preferably in amounts of 0.1 to 20 wt. %, based on the total product. Quantities of 0.2 to 10 wt %, in particular 0.3 to 5 wt %, are particularly preferred. The amount of propellant is based on the total weight of the product and corresponds to the total amount when more than one propellant is used.

In a further embodiment, the problem addressed by the present invention is solved by the use of the cosmetic product as described above in the form of a foam for temporarily shaping and/or setting the shape of keratin-containing fibers, in particular human hair.

In yet another embodiment, the problem addressed by the present invention is solved by means of a method for temporarily shaping keratin-containing fibers, in particular human hair, characterized in that the cosmetic product is applied to the keratin-containing fibers as described above.

It has been found to be preferable that the keratin fibers are not rinsed after the action of the cosmetic product of the first subject matter of the invention, and said product is left on the fiber.

Features relating to preferred embodiments of the first embodiment, which are described above only in this regard, naturally also apply accordingly to the further embodiments as features of preferred embodiments.

The following examples are intended to explain the subject of the present invention, without limiting it in any manner.

### EXAMPLES

The following products 1 to 5 according to the invention and the comparative formulations V1 and V2 were prepared.

| | Ex. 1 | Ex. 2 | Ex. 3 | Ex. 4 | Ex. 5 | V1 | V2 |
|---|---|---|---|---|---|---|---|
| | wt. % | wt. % | wt. % | wt. % | wt. % | wt. % | wt. % |
| | (active | (active | (active | (active | (active | (active | (active |
| Ingredient | substance) | substance) | substance) | substance) | substance) | substance) | substance) |
| Non-fully | 9.2 | 16 | 1 | 1.96 | 1.96 | — | 2 |
| synthetic film- | | | | | | | |
| forming agent | | | | | | | |
| Fully synthetic | 1.0 | 1.2 | 1.1 | 1.1 | — | 8 | 6 |
| film-forming | | | | | | | |
| agent | | | | | | | |
| Shea butter | 7.6 | 3 | 6 | 2.1 | 2.1 | — | — |
| Emulsifier | 1.1 | 1.2 | 1.0 | 1.5 | 1.1 | 1.2 | 1.2 |
| Surfactants | 2 | 7 | 0.5 | 1 | 1 | 2 | 2 |
| Cetearyl | 0.2 | 0.6 | 2.5 | 1.45 | 1.45 | 2.4 | 1.4 |
| alcohol | | | | | | | |
| Fragrance | 0.15 | 0.2 | 0.10 | 0.15 | 0.15 | 0.2 | 0.15 |
| Auxiliaries | 0.5-1.5 | 0.5-1.5 | 0.5-1.5 | 0.5-1.5 | 0.5-1.5 | 0.5-1.5 | 0.5-1.5 |
| Water | up to 100 | up to 100 | up to 100 | up to 100 | up to 100 | up to 100 | up to 100 |
| (demineralized) | | | | | | | |

Conventional pH adjusters, UV stabilizers, dyes, preservatives and thickeners were combined as auxiliaries.

The agents (examples 1 to 5 and the comparative examples V1 and V2) were present in the form of an aerosol.

The formulations according to the invention (examples 1 to 5) and the comparative formulations (V1 and V2) according to the comparative example were applied to subjects for setting the shape of the hairstyle by hand.

In formulations 1 to 5 according to the invention, the hair received a natural shine and a strong but flexible hairstyle hold. In addition, the subjects had a positive hair feel.

In the comparative examples, the product left a dry hair feel, and the subject was therefore not convinced by the product performance despite good hairstyle hold.

1. A cosmetic product for temporarily shaping keratin-containing fibers, the cosmetic product comprising:
at least one non-fully synthetically produced film-forming agent;
a cosmetic carrier; and
at least one care agent,
wherein the care agent is selected from the group consisting of shea butter, mango butter, cocoa butter, avocado butter, shea oil, capuacu butter, illipe nut butter, kokum butter, sal butter, murumuru butter, olive butter, aloe butter, ghee, coconut oil, organic nilotica shea butter, and mixtures thereof.

2. The cosmetic product of claim 1, wherein the care agent is from 0.1 wt. % to 10 wt. % of the total weight of the cosmetic product.

3. The cosmetic product of claim 1, wherein the at least one non-fully synthetically produced film-forming agent comprises a saccharide.

4. The cosmetic product of claim 3, wherein the at least one non-fully synthetically produced film-forming agent further comprises a cellulose, a cellulose derivative, a starch, a modified starch, a pullulan, and/or guar hydroxypropyltrimonium chloride.

5. The cosmetic product of claim 1, further comprising a fully synthetic film-forming agent.

6. The cosmetic product of claim 5, wherein the at least one non-fully synthetically produced film-forming agent and the fully synthetic film-forming agent are from 0.05 wt. % to 30 wt. % of the total weight of the cosmetic product.

7. The cosmetic product of claim 1, wherein the cosmetic product is substantially free of a fully synthetic film-forming agent.

8. The cosmetic product of claim 1, further comprising at least one propellant.

9. The cosmetic product of claim 8, wherein the propellant comprises at least one of N₂O, dimethyl ether, CO₂, nitrogen, air, alkanes having 3 to 5 carbon atoms, propane, n-butane, isobutane, n-pentane, or isopentane.

10. The cosmetic product of claim 8, wherein the amount of propellant is sufficient to generate a pressure of 5 bar to 15 bar in a container containing the cosmetic product.

11. Use of the cosmetic product of claim 1 for temporarily shaping keratin-containing fibers.

12. A method for temporarily shaping keratin-containing fibers, the method comprising:
applying the cosmetic product of claim 1 to the keratin-containing fibers in the form of a foam.

13. The method of claim 12, wherein the cosmetic product is left on the keratin-containing fibers and is not rinsed out.

14. The cosmetic product of claim 1, wherein the keratin-containing fibers are human hair.

15. The cosmetic product of claim 1, wherein the cosmetic product is in the form of a foam.

16. The cosmetic product of claim 2, wherein the care agent is from 1.5 wt. % to 3 wt. % of the total weight of the cosmetic product.

17. The cosmetic product of claim 3, wherein the saccharide comprises a monosaccharide, a disaccharide, a polysaccharide, and/or a biopolymer that is chemically modified.

18. The cosmetic product of claim 6, wherein the at least one non-fully synthetically produced film-forming agent and the fully synthetic film-forming agent are from 0.15 wt. % to 15 wt. % of the total weight of the cosmetic product.

19. The use of claim 11, wherein the keratin-containing fibers are human hair.

20. The method of claim 12, wherein the keratin-containing fibers are human hair.
